(19) United States
(12) Patent Application Publication
(10) Pub. No.: US 2010/0114044 A1
(43) Pub. Date: May 6, 2010

(54) ADHESIVE BODY FITMENT FOR OSTOMY APPLIANE

(76) Inventors: Kathryn E. Cramer, Manasquan, NJ (US); Matthew Dawson, Hillsborough, NJ (US); Tinh Nguyen-DeMary, Milltown, NJ (US)

Correspondence Address: CONVATEC INC., 100 HEADQUARTERS PARK DRIVE, SKILLMAN, NJ, 08558, US

(21) Appl. No.: 12/450,713

(22) PCT Filed: Apr. 8, 2008
(86) PCT No.: PCT/US08/59629

§ 371 Date: Oct. 8, 2009

Related U.S. Application Data
(60) Provisional application No. 60/910,681, filed on Apr. 9, 2007.

(51) Int. Cl. A61F 5/44; A61L 24/00

(52) U.S. Cl. 604/332

(57) ABSTRACT

A body fitment for an ostomy appliance comprises an adhesive wafer including a body-facing side for adhesively contacting the user's body. The adhesive wafer includes a moldable region shapable by the user, and a hole therethrough for allowing the user to manually mold a stomal aperture in the moldable region. The body fitment is provided with the inner peripheral region of the adhesive bordering the hole being pre-shaped with a non-planar configuration, e.g. bent or curled away from the body-facing side of the adhesive, prior to be being molded by the user. The pre-shaped, non-planar, configuration is supported by a release device that initially protects the body-facing side of the adhesive but which is intended to be removed prior to use of the body fitment, to enable the adhesive then to be molded by a user.

The present invention relates to an adhesive body fitment for an ostomy appliance. In particular, the invention relates to a fitment including a moldable adhesive. This fitment may be utilized in a one-piece appliance in which the adhesive body fitment is permanently attached to the appliance, or a two-piece appliance in which the adhesive body fitment is releasably attachable to the appliance.

### BACKGROUND TO THE INVENTION

The adhesive body fitments most commonly used for attaching ostomy appliances to the body are designed to have a generally fixed shape that is predetermined by manufacture. The adhesive body fitment has a central opening that can be cut to enlarge the opening to a desired size to fit a wearer's stoma. The adhesive is pre-printed with concentric guidance lines around the opening for certain standard stoma sizes, and the user uses scissors to cut along the appropriate line to enlarge the opening to the selected size.

U.S. Pat. No. 6,840,924 describes an improved ostomy appliance including a moldable adhesive, in which at least a portion of the adhesive can be manually molded by the wearer, to provide a customized size and shape of opening. The adhesive, initially in a planar configuration, is molded by rolling back the inner peripheral edge of the stoma aperture on the side facing away from the body of the wearer. This alternative way of customizing the wafer offers the prospect of a better fit around the stoma than that obtainable by cutting along standard size guidelines. Achieving a good fit is desirable in order to reduce the exposure of the peristomal skin to stool exiting the stoma. Peristomal skin may be quite sensitive, and vulnerable to irritation or infection when contacted by stool. Stool exiting the stoma may contain digestive juices from the body, and such juices can also attack the peristomal skin resulting in excoriation. However, stomas have different sizes and shapes. The advantage of a moldable adhesive as taught in U.S. Pat. No. 6,840,924 is that the user can mold the adhesive to closely match the exact size and shape of his or her stoma.

In a form in which the appliance of U.S. Pat. No. 6,840,924 is implemented as a two-piece ostomy appliance, the wearer is able to access the moldable adhesive on the non-body-contacting side, when the pouch is separated from the body fitment. This can enable the wearer to shape the adhesive by rolling back the adhesive on the non-body-contacting side. However, when implemented as a one-piece appliance, the wearer can only access the moldable adhesive from the body-contacting side, and before the body fitment is secured to the body. This means that the shape and size of the aperture might not be as accurate as with a two-piece ostomy appliance.

Another example of an adhesive body fitment employing a moldable adhesive is described in U.S. Published Application No. 2006/0184145. This specification teaches an initially planar adhesive wafer that is moldable by rolling back the inner rim of the adhesive away from the body-facing side of the wafer, in a manner almost no different from the aforementioned U.S. Pat. No. 6,840,924. A further example is illustrated in U.S. Pat. No. 6,332,879.

In devising the present invention, the inventors have appreciated that it would be desirable to enhance the ease of use of an ostomy appliance including a moldable adhesive, especially but not exclusively, a one-piece appliance.

### SUMMARY OF THE INVENTION

One aspect of the present invention provides an adhesive body fitment for attaching an ostomy appliance to a wearer's body. The body fitment comprises an adhesive wafer including a moldable region having a starter hole therethrough for allowing a user to manually mold a stomal aperture in the moldable region. The inner peripheral region of the adhesive bordering the hole is preshaped to have, prior to molding by the user, a non-planar configuration. The non-planar configuration may enable the user more easily to begin molding the adhesive into a desired form. The non-planar configuration may present the inner edge of the adhesive bordering the hole, offset towards one side of the wafer.

In one form, the non-planar configuration is a bulge or protruberance away from the body-facing side of the wafer. The adhesive at the starter hole is bent to be in front of (i) the rearmost surface of the adhesive and/or (ii) a generally planar portion of the adhesive at the outer periphery of the adhesive. Such a configuration of the moldable adhesive at, or near, the starter hole can facilitate rolling of the adhesive in a frontward direction away from the body-facing side of the wafer. The configuration pre-disposes the adhesive to be more easily molded than, for example, were the adhesive to have a purely planar shape. The invention is especially suitable for use in a one-piece ostomy appliance in which access to the adhesive, to fold back the adhesive away from the body-facing side, is restricted. However, the invention also finds use in a two-piece appliance, where ease of molding is also enhanced, for example for non-dextrous or elderly users.

Additionally or alternatively, the wafer may further comprise a release device that supports the shape of the non-planar configuration. The release device covers and/or protects the adhesive surface of the wafer, and is intended to be removed from the adhesive before the wafer is placed against the skin. The release device includes a generally stiff or fixed-shape support that defines the non-planar configuration of the moldable adhesive.

The term “moldable” as used herein is intended to mean generally that the adhesive may be any of deformed or extruded or reshaped by hand. In a preferred form, the moldable adhesive portion is moldable as a deformable adhesive sheet, meaning that the wafer retains generally sheet-like properties (in contrast to an extrudable form, for example), and the adhesive wafer may be molded by bending or flexing the adhesive wafer in the moldable region, or by rolling the adhesive wafer back on itself. Such a moldable wafer is especially configured for allowing the stomal aperture in the adhesive to be enlarged to a customized size and/or shape. The adhesive wafer may optionally have a spring-back property such that the adhesive may tend to spring back partly towards its initial shape. Such a spring-back property may be useful for sealing around the stoma.

In one embodiment, the ostomy appliance is a one-piece item, in which the body fitment is permanently attached to the ostomy pouch. The term “permanently” as used herein is intended to mean that the body fitment is attached or attachable to the pouch in such a way that the fitment is not easily separable from the pouch without damage and/or is never intended to be separable from the pouch during normal use.

While features believed to be of special significance have been identified above claim protection may be sought for any patentable feature described herein and/or illustrated in the drawings.

### BRIEF DESCRIPTION OF THE DRAWINGS

FIG. 1 is a schematic cross-sectional view through a first embodiment of ostomy pouch, in the form of a one-piece appliance.

FIG. 2 is a schematic perspective view of the release member from the pouch of FIG. 1.

FIG. 3 is a schematic cross-sectional view showing use of the first embodiment when the release member is removed.

FIG. 4 is a schematic cross-sectional view through a second embodiment of ostomy pouch in the form of a one-piece appliance having a shape-defining member.

FIG. 5 is a schematic cross-sectional view through a third embodiment of ostomy pouch in the form of a two-piece appliance.

### DETAILED DESCRIPTION OF THE PREFERRED EMBODIMENTS

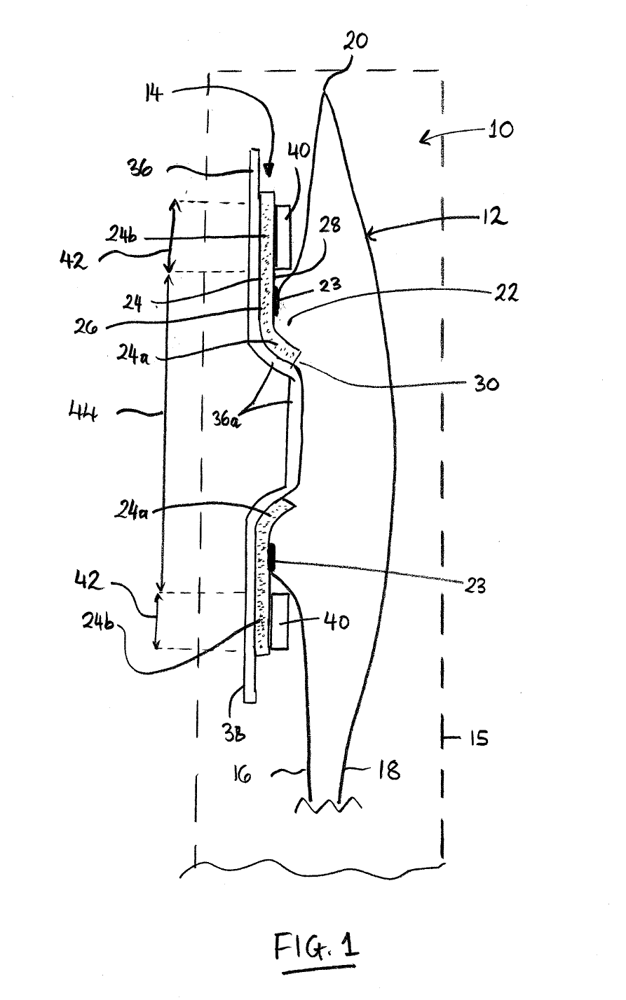

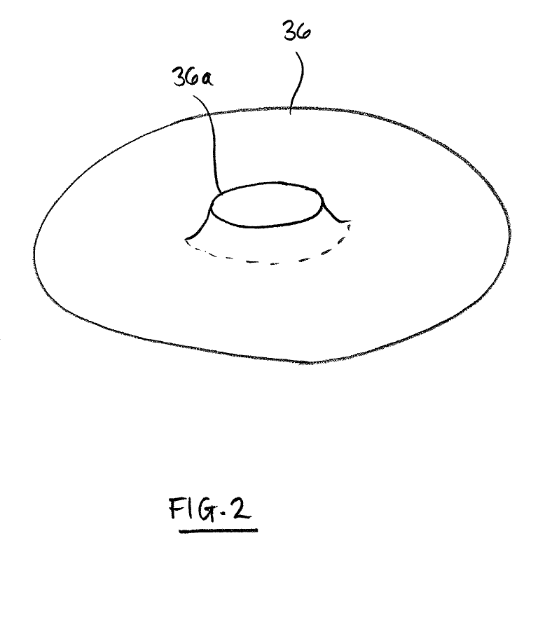

Referring to FIGS. 1 and 2, a first embodiment illustrates a one-piece ostomy appliance 10 comprising an ostomy pouch 12 and a body fitment 14 permanently attached to the ostomy pouch 12. The ostomy pouch 12 may be any of a colostomy pouch, a urostomy pouch and an ileostomy pouch. The ostomy appliance 10 represents ostomy equipment that is optionally supplied to a user in a package 15. The package 15 can take various forms, for example, a sealed bag or a blister pack. In the present embodiment, the package 15 is sterile inside, and is hermitically sealed in a closed condition.

The ostomy pouch 12 includes a rear wall 16 and a front wall 18. The front and rear walls 16, 18 are made of flexible plastics film that is generally impermeable to liquid and gas. A suitable film includes, for example, a laminate of one or more layers of ethylene vinyl acetate (EVA), and one more layers of a gas barrier material, such as poly(vinylidene chloride) (PVDC). The walls 16 and 18 are welded together around a mutual periphery 20.

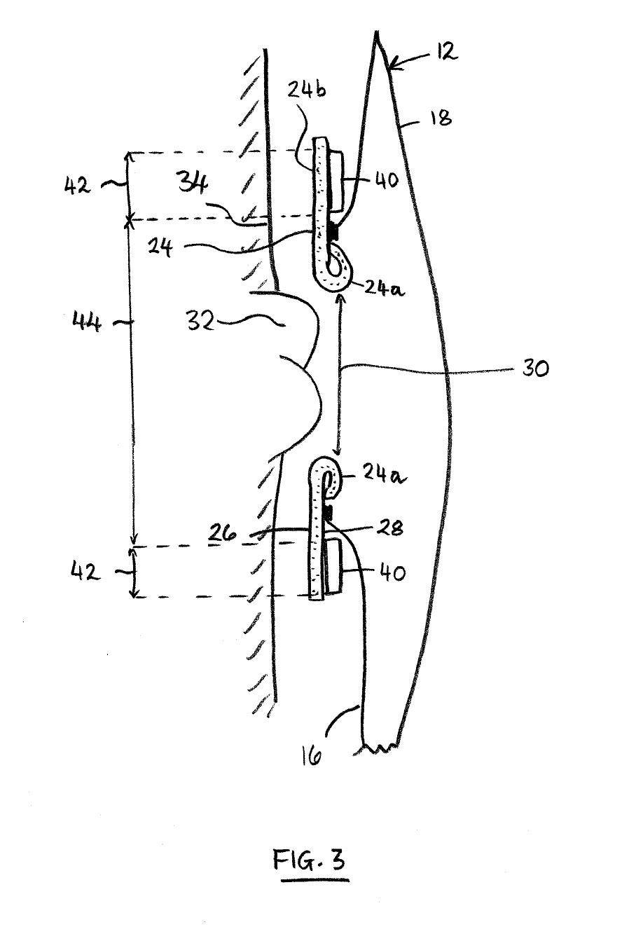

The rear wall 16 has a stomal aperture 22 around which the body fitment 14 is attached to the rear wall 16. The body fitment 14 is attached, for example, by a suitable strong adhesive or weld in a region 23 surrounding the stomal aperture 22. The body fitment 14 includes a wafer of a skin compatible adhesive 24. The adhesive 24 generally has a body-facing side 26 for facing towards and contacting the skin, and a non-body-facing side 28 that faces away from the skin, and instead faces towards the interior of the ostomy pouch 12. In this embodiment, the adhesive 24 is of a moldable type, in which at least a portion of the adhesive 24 (such at least an inner peripheral region 24a bordering a starter hole 30), is moldable or shapeable by manual manipulation, to enable the size and/or the shape of the hole 30 to be customized to match the size and/or shape of the ostomate's stoma 32 (FIG. 3). A close or snug fit around the stoma 32 is highly desirable, in order to protect the ostomate's peristomal skin 34 from irritation and excoriation by contact with stool exuding from the stoma 32. The adhesive 24 may, for example, be of either of the types described in the aforementioned U.S. Pat. No. 6,840,924, and U.S. Published Application No. 2006/0184145. Such an adhesive 24 is configured in a moldable wafer form to enable the adhesive 24 to be shaped while retaining a sheet-like form. In one form, the adhesive 24 comprises a laminate structure including plural layers of adhesive, and/or at least one layer of adhesive and at least one layer of plastics film. One or both of the pouch walls 16, 18 (e.g., the front wall 18) optionally is transparent, or includes a transparent portion, so that the user may more easily see the shape of the hole 30, and the fit of the adhesive 24 around the stoma 32, when in use.

As best seen in FIG. 1, the inner peripheral region 24a of the moldable adhesive 24 surrounding the starter hole 30 is pre-shaped during production of the body fitment 14 or the ostomy appliance 10, to have a non-planar configuration. In this embodiment, the inner peripheral region 24a is bent or at least partly curled so as to bulge or protrude frontwardly, i.e., away from the body-facing side 26 of the adhesive. The inner peripheral region 24a has a frusto-conical, or flared, or at least partly curled, shape. The inner peripheral region 24a therefore lies more in front of a generally planar region 24b of the adhesive 24 near the outer periphery of the wafer.

The non-planar configuration of the adhesive 24a is supported by a release device in the form of a release member 36 that is supplied with the ostomy appliance 10. The release member 36 covers and protects the adhesive surface on the body-facing side 26 of the wafer. The release member 36 comprises a support 36a in the form of an elevated projection to define and support the non-planar configuration of the adhesive 24a. The base of the support 36a has a larger transverse dimension (e.g., diameter or radius) than that of the starter hole 30, such that the inner peripheral region 24a is supported in the non-planar shape defined by the support 36a. The release member 36 is made of, or is at least coated with, a release material such a silicone that does not stick strongly to the adhesive 24 of the wafer. For example, the release member 36 may be made of silicone coated card, silicone coated plastics, or silicone coated paper. At least the support 36a of the release member 36 is relatively stiff, so as to maintain a relatively well defined shape of the adhesive 24a around the hole 30. The remainder of the release member 36 may be more flexible. Alternatively, it may also be relatively stiff so as to control the shape of the adhesive 24 generally throughout the wafer. The release member 36 may include a radially projecting tab 38, or an annularly projecting periphery 38, for enabling the release member 36 easily to be gripped and peeled from the adhesive 24 prior to use of the ostomy appliance 10. Although in this embodiment the release device is formed by a single, one-piece release member 36 of which the support 36a is an integral part, in other embodiments, the support may be a separate element. For example, the release device may comprise a release sheet and a support non-integral with the release sheet.

In order to use the ostomy appliance 10, the user removes the release member 36, to expose the body-facing side 28 of the adhesive 24. Even unsupported, the inner peripheral portion 24a of the adhesive 24 will tend to retain, at least for a period of time, the non-planar configuration previously imparted by the support 36a of the release member 36. As illustrated in FIG. 3, the hole 30 in the adhesive 24 can be expanded to a customized shape and/or size by bending and rolling the adhesive 24a back on itself around the hole 30, on the non-body-facing side 28 of the adhesive 24. The non-planar configuration of the adhesive 24 makes it easier for a user to begin manipulating and rolling the adhesive 24 against the non-body-facing side 28, even though the user has to perform this task by inserting his or her fingers through the hole 30 from outside the ostomy appliance 10, i.e., the user only has access from the body-facing side 26 of the adhesive 24. Since the adhesive region 24a is already bent away from the body-facing side 26 of the adhesive 24, this promotes molding and folding of the adhesive 24 against the non-body-facing side 28. The non-planar configuration can thus make molding the adhesive 24 easier than were the adhesive 24 (especially the inner peripheral region 24a) only to have a planar shape. If the front wall 18 is transparent, or includes a transparent portion, this may aid the user in seeing the shape that is molded. The rolled back surface of the adhesive 24 sticks to the non-body facing surface 28 (or a sheet provided thereon), thus forming a stable molded, rolled-back shape. As mentioned above, the adhesive 24 may have slight spring back characteristics if desired.

Once the inner peripheral region 24a of the adhesive 24 has been molded to obtain the desired custom shape and size of the hole 30, the ostomy appliance 10 is pressed against the peristomal skin 34, with the stoma 32 aligned with the hole 30, in order to mount the ostomy appliance 10 on the body.

In the first embodiment, the adhesive 24 includes a generally planar portion 24b. If desired, the planar portion 24b of the adhesive 24 may be reinforced by a shape defining member 40 mounted against the non-body-facing side 28. The optional shape defining member 40 may be in the form of an annular ring of a flexible, but relatively stiff, material (being stiff in comparison to the adhesive 24). The shape defining member 40 thus defines a generally annular non-shapeable (or constrained shape) zone 42 in the adhesive region 24b that is supported by, and has a shape controlled by, the shape defining member 40. The wafer further includes a shapeable zone 44 inside the non-shapeable zone 42. The shapeable zone 44 includes the inner peripheral region 24a of adhesive 24 having the non-planar configuration, and being moldable or shapable once the release member 36 has been removed.

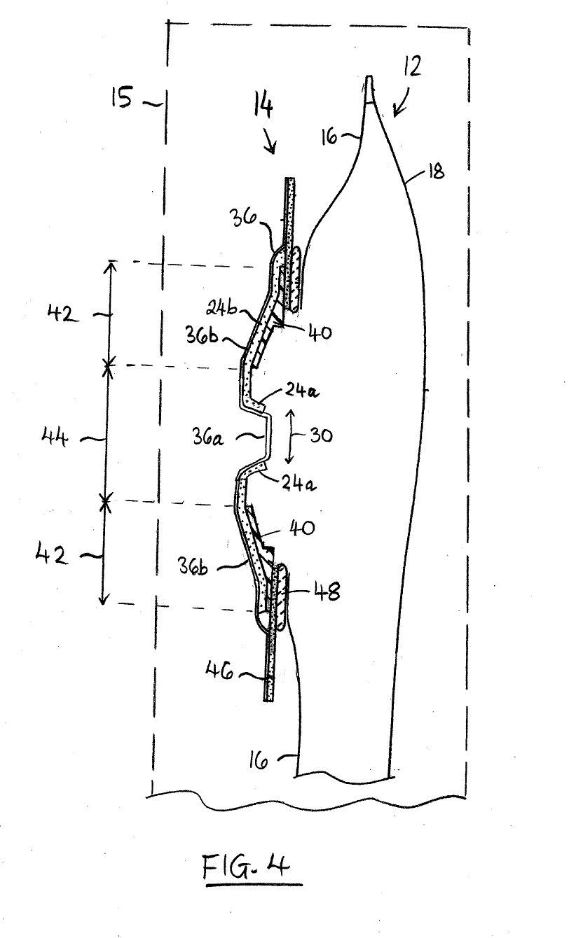

In the second embodiment illustrated in FIG. 4, the shape defining member 40 itself has a non-planar shape, to define a bulge in the adhesive 24 towards the body-facing side 26. Such a bulged shape may be suitable for an ostomate who has a recessed stoma. The adhesive 24 includes a rearwardly bulging portion 24b having a shape defined by the shape defining member 40, and a frontwardly protruding or re-entrant portion, being the inner peripheral portion 24a around the hole 30. The inner peripheral portion 24a lies more in front than the rearmost portion of the adhesive 24. The release member 36 has a corresponding shape including an outer dish portion 36b to match the shape of the shape-defining member 40, and a re-entrant support projection 36a, similar to the support 36a of the first embodiment, in order to define the frontward protruding shape of the inner peripheral portion 24a of adhesive 24.

When the release member 36 is removed, the inner peripheral portion 24a of adhesive 24 is moldable (shapable zone 44) to enable the user to form the hole 30 into a desired size and shape, as in the first embodiment. The protruberance of the inner peripheral portion 24a bent away from the body-facing side 26 of the adhesive 24 promotes molding of the adhesive 24 by rolling against the non-body-facing side 28. The portion 24b of the adhesive 24 supported by the shape-defining member 40 retains a generally well-defined, bulged shape controlled by the shape-defining member 40 (non-shapeable or constrained shape zone 42).

In the illustrated embodiment, the body fitment 14 further comprises an adhesive patch 46 made, for example, or fabric or microporous sheet, for adhering to the skin around the adhesive 24. The adhesive surface of the patch 46 may initially be covered by the release member 36, or by a separate release sheet that the user can peel from the patch 46 prior to adhering the ostomy appliance 10 to the skin. The ostomy appliance 10 further comprises an annular reinforcing ring 48 for stabilizing the shape of the stomal opening 22 of the ostomy pouch 12. These elements may also be included in the other embodiments, as desired.

As illustrated by the first and second embodiments, the invention is especially suitable for enhancing the use of one-piece ostomy appliances, by pre-shaping the inner peripheral portion 24a of the adhesive 24 to protrude or project away from the body-facing side 26 of the adhesive 24, so that it is more easily molded against the non-body-facing side 28 of the adhesive 24. However, as illustrated by the following third embodiment, the invention also finds use when implemented in a two-piece appliance.

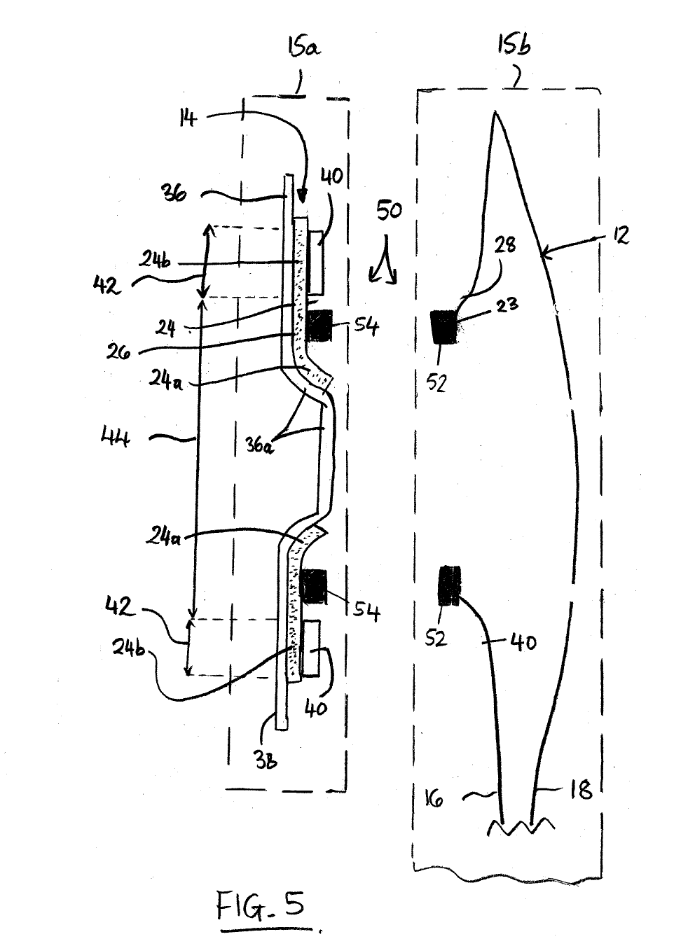

Referring to FIG. 5, in the two-part ostomy appliance 10, the ostomy pouch 12 is releasably attachable to the body fitment 14 by means of an ostomy coupling 50. The ostomy coupling 50 includes a pouch-side component 52 carried on the ostomy pouch 12 and releasably attachable to a fitment-side component 54 carried on the body fitment 14. The two components 52 and 54 may be releasably attachable by means of a peelable adhesive, or by a mechanical interlocking or interference engagement.

In the third embodiment, the body fitment 14 includes the wafer of adhesive 24 including the non-planar configuration of the inner peripheral portion 24a of the adhesive around the starter hole 30. The non-planar configuration of the adhesive 24 pre-disposes the inner peripheral portion 24a to be more easily molded by rolling or folding the adhesive 24 against the non-body-facing side 28. In particular, the edge of the adhesive 24 around the opening 30 is bent away from the body-facing side 26 of the adhesive 24. The pre-disposition of this edge of the adhesive 24 makes it easier, especially for elderly or non-dextrous users, to manipulate the adhesive 24 than were the inner peripheral portion 24a to have a flat or planar shape. Although the third embodiment is illustrated to have an adhesive shape similar to the first embodiment, it will of course be appreciated that the adhesive 24 may be given a bulged shape as in the second embodiment, by using a non-planar shape defining member. In the third embodiment, the entire ostomy appliance 10 may be supplied in a single package (not shown) similar to that of the first embodiment, or the body fitment 14 and the ostomy pouch 12 may be supplied in separate packages 15a and 15b.

It will be appreciated that the foregoing description is illustrative of preferred forms of the invention, and that many modifications, improvements and equivalents are possible that are within the scope of the claimed coverage of the invention.

We claim:

1. An adhesive body fitment for attaching an ostomy appliance to a wearer's body, the body fitment comprising:
a. an adhesive wafer including a body-facing side for adhesively contacting the wearer's body, the adhesive wafer further including:
a moldable shapable region having a hole therethrough for allowing manual molding of a stomal aperture in said moldable region, the inner peripheral region of the adhesive bordering the hole having a non-planar configuration; and
b. a removable release device fitted to the body-facing side of the adhesive wafer, the release device including a support for maintaining the non-planar configuration of the inner peripheral region of the adhesive bordering the hole, until said release device is removed prior to molding of the moldable region of the adhesive.

2. The adhesive body fitment according to claim 1, wherein the inner peripheral region of the adhesive bordering the hole bends to extend in a direction away from said body-facing side of said adhesive, and said support is shaped for supporting said inner peripheral region of the adhesive in the bent configuration.

3. The adhesive body fitment according to claim 2, wherein the inner peripheral region of the adhesive bordering said hole at least partly curls away from said body-facing side of the adhesive, and said support is shaped for supporting said inner peripheral region of the adhesive in the at least partly curled configuration.

4. The adhesive body fitment according to claim 3, wherein the inner peripheral region of the adhesive has a configuration selected from frusto-conical; flared; partly curled, and said support is shaped for supporting said inner peripheral region in said configuration.

5. The adhesive body fitment according to claim 1, wherein the adhesive wafer has a generally uniform thickness.

6. The adhesive body fitment according to claim 1, wherein the moldable region of the adhesive is integral with the remainder of the adhesive wafer.

7. The adhesive body fitment according to claim 1, wherein the release device comprises a single member including said support.

8. The adhesive body fitment according to claim 1, wherein the release device is made of, or is coated with, a material to which the adhesive of the wafer does not strongly adhere.

9. The adhesive body fitment according to claim 1, wherein the adhesive wafer further includes a constrained-shape region surrounding the moldable region, the constrained-shape region being (i) substantially less shapeable than the shapeable region; or (ii) of substantially fixed shape.

10. The adhesive body fitment according to claim 9, further comprising a shape-defining member provided on the opposite side of the adhesive wafer to said body-facing side, said shape defining member constraining the shape of said constrained-shape region of the adhesive.

11. The adhesive body fitment according to claim 10, wherein the shape-defining member is substantially planar.

12. The adhesive body fitment according to claim 10, wherein the shape-defining member is substantially non-planar.

13. An ostomy appliance comprising an adhesive body fitment for attaching the ostomy appliance to a wearer's body, the body fitment comprising:
a. an adhesive wafer including a body-facing side for adhesively contacting the wearer's body, the adhesive wafer further including:
a moldable shapable region having a hole therethrough for allowing manual molding of a stomal aperture in said moldable region, the inner peripheral region of the adhesive bordering the hole having a non-planar configuration; and
b. a removable release device fitted to the body-facing side of the adhesive wafer, the release device including a support for maintaining the non-planar configuration of the inner peripheral region of the adhesive bordering the hole, until said release device is removed prior to molding of the moldable region of the adhesive.

14. The ostomy appliance according to claim 13, wherein the ostomy appliance is a one-piece appliance.

15. The ostomy appliance according to claim 13, wherein the ostomy appliance is a two-piece appliance.

16. An assembly comprising:
a. a package for supply to a user; and
b. ostomy equipment contained in said package, said ostomy equipment including an adhesive body fitment for attaching an ostomy appliance to a wearer's body, the body fitment comprising:
an adhesive wafer including a body-facing side for adhesively contacting the wearer's body, the adhesive wafer further including a moldable shapable region having a hole therethrough for allowing manual molding of a stomal aperture in said moldable region, and at least an inner peripheral portion of the moldable region bordering said hole being pre-shaped with a non-planar configuration in which said adhesive extends in a direction away from said body-facing side of the adhesive, to facilitate molding.

17. The assembly according to claim 16, wherein the package is sterile inside.

18. The assembly according to claim 16, wherein the package is hermetically sealed in a closed condition.

19. The assembly according to claim 16, wherein the ostomy equipment comprises a one-piece ostomy appliance to which said adhesive body fitment is permanently attached.

20. The assembly according to claim 16, wherein the ostomy equipment is a two-piece ostomy appliance in which said body fitment is a releasably attachable component.

21. A method for adhering an ostomy pouch to a wearer, the steps comprising:
providing an ostomy pouch with a body fitment wherein the body fitment includes an adhesive wafer having a body-facing side for adhesively contacting the wearer's body, the wafer including a moldable shapeable region having a hole therethrough, and a removable release cover fitted to the body-facing side of the adhesive wafer around the hole;
molding the moldable shapeable region of the wafer with said removeable release cover in place; and
subsequently removing the removable release cover from said adhesive wafer.
